(19) United States
(12) Patent Application Publication
(10) Pub. No.: US 2021/0315483 A1
(43) Pub. Date: Oct. 14, 2021

(54) Method for Obtaining a Breath Sample of a Test Person and Device

(71) Applicant: Robert Bosch GmbH, Stuttgart (DE)

(72) Inventor: Franz Laermer, Weil Der Stadt (DE)

(21) Appl. No.: 17/260,012

(22) PCT Filed: Jul. 23, 2019
(86) PCT No.: PCT/EP2019/069734

§ 371 Date: Jan. 13, 2021

(30) Foreign Application Priority Data
Jul. 25, 2018 (DE) 10 2018 212 411.6

(51) Int. Cl. A61B 5/097; A61B 10/00; A61B 5/08; A61B 5/00

(52) U.S. Cl. CPC A61B 5/097; A61B 10/0051; A61B 2010/0087; A61B 5/4848; A61B 5/082

(57) ABSTRACT

The disclosure relates to a method and a device for obtaining a breath sample of a test person, wherein the test person inhales a droplet mist which is preferably colder than the ambient temperature. The material exhaled and/or ejected by the test person is collected as a breath sample in one or more collection vessels.

The present invention relates to a method for obtaining a breath sample from a test person and to a apparatus provided for obtaining a breath sample from a test person.

### PRIOR ART

It is known to collect a breath sample from a test person or patient and to obtain, from said sample, information concerning the state of health of said test person or patient. For example, various proteins or other substances (biomarkers) can be isolated from an obtained breath sample and can be used to draw conclusions concerning an existing cancer of the lungs, a metabolic disorder in the lungs, an infection of the lungs, or also existing systemic diseases, for example cancers or general infections. For example, tuberculosis bacteria can be isolated from the obtained breath sample in the context of tuberculosis screening. Moreover, certain bacteria or viruses can be isolated from a breath sample, further analyzed and, if appropriate, identified for the diagnosis of lung infections or lung inflammations as what are called respiratory infections. It is also possible to isolate genetic material, in particular DNA, from a breath sample in order to be able to draw conclusions as regards any viruses, bacteria, fungi or tumor cells that may be present. Analytical methods required for this, in particular molecular biological methods, have become established. Such analytical methods are also open to automated processing, such that they can be used, for example, in the context of a screening process for a larger population group. In addition, the evaluation of breath samples can be used in the optimization of medical therapies, for example in the monitoring of antibiotic therapy for a bacterial infection, or in the monitoring and optimization of cancer therapy. Furthermore, the evaluation of a breath sample can be used in the stratification of patients, i.e. for assessing the risk of a disease. The general advantage of examining breath samples is that the breath sample can generally be obtained in a very simple (non-invasive) manner that does not place a burden on the patient.

Breath condensate collection has often been used hitherto to obtain a sample from the breath. For this purpose, the moisture contained in the exhaled air is condensed out on cold trap surfaces. However, it is mainly small molecules that are dissolved in the breath condensate, and these are meaningful only in particular cases. For example, breath condensate is suitable for the determination of H₂O₂, which is tested in the context of monitoring chronic obstructive pulmonary diseases (COPD). There are also approaches which involve collecting aerosols (very small water droplets released in the lungs during exhalation) and isolating them, for example, together with a moisture condensate.

### DISCLOSURE OF THE INVENTION

### Advantages of the invention

The invention makes available a method for obtaining a breath sample from a test person, in which method the test person inhales a droplet mist. The droplet mist is preferably colder than the ambient temperature. The material then exhaled and/or coughed up by the test person is collected as a breath sample. With this method it is possible in particular to obtain diagnostically interesting and meaningful materials from the lungs and to use these materials for an analysis. The droplets of the inhaled mist interact with the inner surfaces of the bronchial system, resulting in a size-independent exchange with the substances on the surfaces. This means that the water droplets absorb material from the surfaces of the bronchial system, for example salts, bacteria, fungal spores, viruses, mobile cancer cells, DNA, RNA, proteins, etc. At the same time, the contact with the cold water droplets, or the water droplets at least subjectively perceived to be cold, causes a mild cough stimulus, such that the water droplets charged with the sample material can be quickly coughed up again and collected. The temperature of the droplet mist is preferably below ambient temperature. Ambient temperature is generally to be understood as the temperature in closed rooms in which the method is used, i.e. room temperature (approximately 18 to 22° C.). The temperature of the droplet mist is preferably between 0° C. and 15° C. In order to generate the subjective sensation of a cold droplet mist, the nebulization or droplet formation by the associated evaporative cold may in some cases be sufficient on its own. In the following, therefore, the droplet mist is also referred to as a cold droplet mist. Compared to conventional methods, the method described has the considerable advantage that substantially more material can be obtained from the lung surfaces, in particular also larger particles, which are very valuable from the diagnostic point of view. By means of the triggered cough stimulus, it is even possible for macroscopically visible particles and solid components to be obtained from the surfaces of the lungs. With the method described here, it is therefore possible to collect much more interesting sample material than, for example, with conventional breath condensate collection.

In addition to triggering a mild cough stimulus, the described method has the further advantage that the low temperature of the water droplets means that a considerable proportion of the cold water droplets as such also leave the lungs together with the coughed-up air stream and do not evaporate during their stay in the airways. Water vapor is less suitable as a carrier for biological sample material, since only very small molecules can be absorbed. By contrast, much more valuable analytical sample material can be obtained via the droplet mist of the method described here.

The method described here is suitable, for example, for use in the context of tuberculosis screening. In the case of tuberculosis screening, a sputum sample is usually taken from the patient, and the bacteria to be detected are possibly located in the sputum. Liquefying the generally very viscous and sticky sputum, and isolating the bacteria possibly contained therein, is difficult and expensive, and therefore screening on the basis of sputum samples is generally difficult in larger groups of patients. Since coughing is brought about in a controlled manner in the method according to the invention and, in addition, the droplet mist provides a transport mechanism for taking up tuberculosis bacteria and for transporting these out of the lungs, the described method is particularly suitable for tuberculosis screening.

In a preferred embodiment of the method, the droplet mist is generated using an ultrasonic atomizer known per se. The generation of the mist by ultrasound has the particular advantage that the generation of the droplets does not require heating of the liquid that is to be atomized. In the ultrasound treatment itself, there is only slight heating of the liquid that is to be atomized. However, the atomization and droplet shape of the liquid, with the associated evaporative cold, is in some cases sufficient to create a subjective sensation of coldness and to bring about a cough that is triggered by this. The sensation of coldness is particularly preferably increased by cooling the generated mist and/or the liquid that is to be atomized. In a very simple and practical embodiment, for example, ice can be placed in the liquid that is to be atomized. In another embodiment, a Peltier element or another cooling element can be provided, for example, which cools the liquid that is to be atomized and/or cools the mist itself.

Apparatuses for ultrasonic atomization are known per se and are available at reasonable cost. Suitable apparatuses are installed, for example, in so-called home inhalers, which can in principle be used for the method described here.

In the method described here, provision is made that the droplet mist is firstly inhaled, and then the exhaled and/or coughed-up material is collected. For this purpose, a suitable apparatus can be provided via which the droplet mist is first of all inhaled. The exhaled flow of air is then blown back into the apparatus and is diverted within the apparatus, for example with the aid of a check valve, to a suitable collecting vessel. During the collecting process, the exhaled flow of air or the exhaled and/or coughed-up material is preferably guided through a filter (e.g. a filter frit) to which the material to be analyzed is bound. For example, bacteria, viruses, fungi, fungal spores, DNA, RNA and/or proteins contained in the exhaled flow of air can be bound to a filter and collected, before the material is further processed and, for example, used for immunological or molecular biological analysis. Processes that are open to automation can advantageously be used for an analysis, for example lab-on-a-chip systems which are particularly suitable, especially for screening methods or other methods with a high throughput of samples.

The invention further comprises an apparatus for obtaining a breath sample from a test person. Here, the apparatus comprises a device for generating a droplet mist, the temperature of which is preferably below the ambient temperature. Furthermore, a device is provided for the aspiration of the droplet mist by the test person and for collecting exhaled and/or coughed-up material. The droplet mist is preferably generated using ultrasound technology. For this purpose, in addition to the actual ultrasound transmitter, a storage vessel is provided for storing the liquid that is to be atomized. The latter is in particular an aqueous solution, for example water. If necessary, further additives can be contained in the aqueous solution, for example dissolved common salt (NaCl), in particular in a physiological or isotonic concentration of about 0.9 g/l water (154 mmol/l). Such a physiological saline solution has the same osmotic pressure as blood, so that the droplets of the mist based on the saline solution are not absorbed or resorbed by the lungs and are able to leave the lungs again particularly efficiently when coughed up.

The storage vessel for storing the liquid that is to be atomized expediently has an air inflow opening and a droplet mist outlet opening. The actual generation of mist, or atomization, is carried out with an ultrasound transmitter known per se. With the ultrasound generator, for example, the water supply in the storage vessel can be slowly atomized. Furthermore, a cooling means is preferably provided in the device for generating the droplet mist. For this purpose, a Peltier cooling element can be provided which cools the liquid supply in the storage vessel. In a particularly simple but nonetheless functional embodiment of the apparatus, the liquid that is to be atomized can be cooled by one or more ice cubes, for example. The supply of liquid that is to be atomized is preferably kept at a temperature in the range between 0° C. and 15° C., in particular between 0° C. and 5° C., since the triggered cough stimulus is strengthened by these temperature ranges. The requirements for the cooling of the liquid supply or for the cooling of the droplet mist may also be dependent on the ambient conditions. In locations where there is a high ambient temperature (e.g. Africa), a sufficiently high cooling capacity is advisable in order to ensure the coldness of the droplet mist.

The ultrasound transmitter and the cooling device can, for example, be operated electrically, for example by means of a mobile battery operation or also a mains operation.

The storage vessel itself can be a disposable article, for example a corresponding plastic vessel. The design of the storage vessel and of other parts of the apparatus as disposable articles has the particular advantage that the vessel and the other parts can be disposed of after use, and there is no risk of persons later using the apparatus and becoming infected. Special disinfection measures can therefore be omitted.

The device for the aspiration of the droplet mist and for collecting the exhaled/coughed-up material can be equipped with a check valve, such that the exhaled flow of air with the exhaled and/or coughed-up material can be diverted into one or more collecting vessels. Furthermore, at least one filter is preferably provided in the collecting vessel or collecting vessels, the filter being provided for binding or for holding back the exhaled and/or coughed-up material. The filter can be, for example, an SiO₂ frit or another suitable filter material with which in particular bacteria, fungi, fungal spores, viruses, cells, DNA, RNA and/or proteins can be held back. Filters made of porous Teflon, for example porous Teflon films, are particularly suitable. The pore sizes of such filters are expediently so small that pathogens or other substances that are to be examined in the breath sample are held back. Suitable pore sizes are in particular in the range from 0.01 to 10 μm, in particular from 0.2 to 5 μm, preferably from 0.5 to 2 μm. For example, filter materials that are used in the context of sterile filtration and that can be obtained as commercially available bacterial filters or pathogen filters are suitable. The choice of suitable filters may also be made dependent on the material that is to be analyzed. For example, certain filters (e.g. 5 to 10 μm pore size) are suitable for the detection of bacteria (e.g. tuberculosis bacteria) or cells, while other filters are suitable for the detection of certain proteins or DNA or RNA (e.g. pore size 0.1 to 1 μm). Filters with a pore size of 0.5 to 2 μm are suitable for many applications. The use of film-like filter materials has the particular advantage that the films can be used over a large surface area, for example by flanging on a funnel equipped with the film. This lowers the respiratory resistance when coughing, making use more pleasant and easier for the test person. For the further analysis of the breath sample, the material to be examined can be rinsed off, for example, from the flat filter, e.g. in a lab-on-a-chip system.

It is particularly preferable that the device for the aspiration of the droplet mist and for collecting exhaled and/or coughed-up material consists of one or more disposable articles. The collecting vessels can, for example, be conventional Eppendorf® filter vessels or filter frits. The rest of the device for the aspiration of the droplet mist and for collecting or filtering the exhaled and/or coughed-up air flow is preferably provided for single use, in order to avoid contamination with possibly infectious material. By using appropriate disposable articles, it is also not necessary, for example, to design the check valve to be impermeable to microbes, since contamination is ruled out by the material being disposed of after use. The articles in question can be manufactured, for example, as plastic parts, which can be made available very cost-effectively and which can be readily adapted to the particular requirements. Provision can also be made, for example, that the storage vessel for storing the liquid that is to be atomized and the device for the aspiration of the droplet mist and for collecting exhaled and/or coughed-up material can be made available as a one-piece article.

Further features and advantages of the invention will become clear from the following description of illustrative embodiments in connection with the drawings. The individual features can be implemented individually or in combination with one another.

In the drawings:

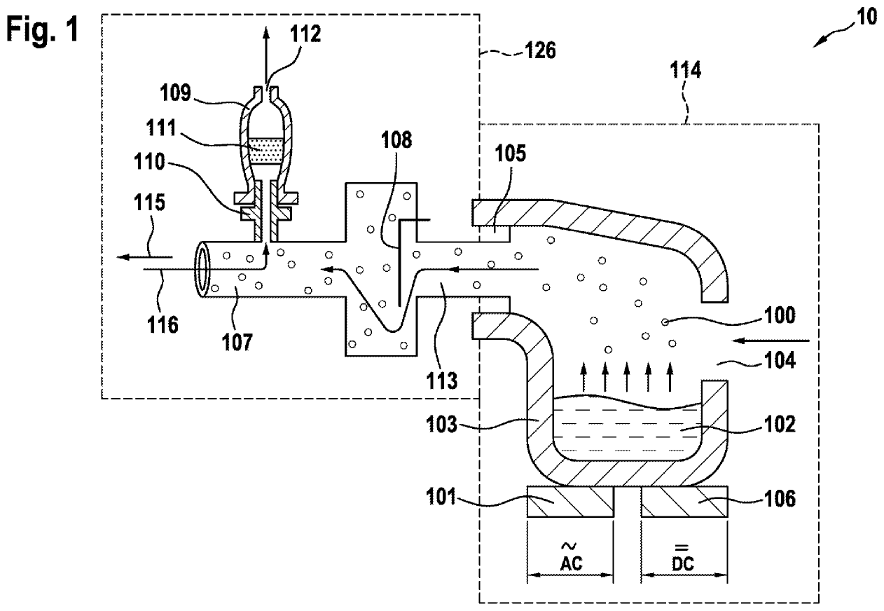

FIG. 1 shows a schematic sectional view of an apparatus for obtaining a breath sample from a test person, and

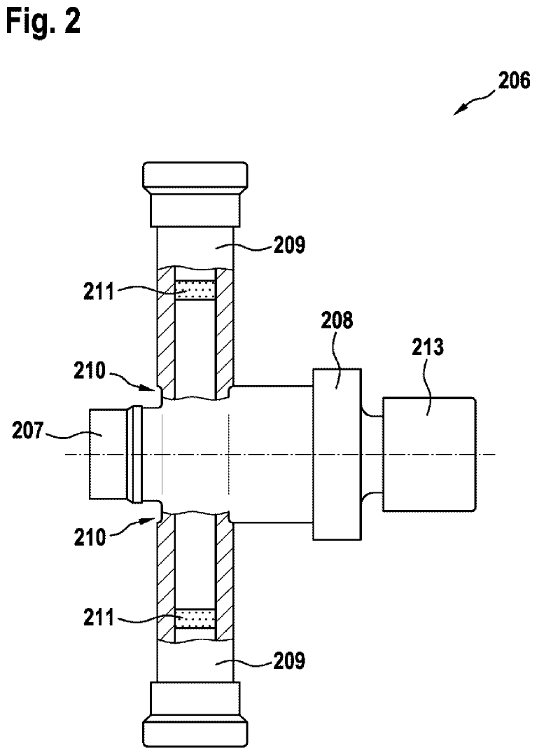

FIG. 2 shows a view of a device for the aspiration of a droplet mist and for collecting the exhaled and/or coughed-up material, as part of the apparatus according to the invention.

### DESCRIPTION OF ILLUSTRATIVE EMBODIMENTS

FIG. 1 shows in schematic form the components of an apparatus 10 for obtaining a breath sample. The apparatus 10 comprises a device 114 for generating a droplet mist 100. In this embodiment, the droplet mist is generated by means of an ultrasound transmitter 101. The liquid 102 that is to be atomized, in particular an aqueous liquid or pure water (tap water), is located in a storage vessel 103. By subjecting the storage vessel 103 to ultrasound using the ultrasound transmitter 101, which in this example is operated with a high-frequency alternating current (AC), the droplet mist 100 is generated in the storage vessel 103. The storage vessel 103 comprises an air inflow opening 104 and a droplet mist outlet opening 105. The air enters through the air inflow opening 104, here indicated by an arrow, and the droplet mist 100 leaves the storage vessel 103 through the droplet mist outlet opening 105, again indicated by an arrow. For the method according to the invention, a droplet mist is used whose temperature is preferably below the ambient temperature and which triggers a mild cough stimulus in the test person when the droplet mist is inhaled. The cold droplet mist can be generated in different ways. In this embodiment, a Peltier cooling element 106 is provided which, for example, can be operated with direct current (DC). By means of the cooling element 106, the aqueous solution or the water 102 in the storage vessel 103 is cooled, such that the formed droplet mist 100 is also cold. In another embodiment, it is possible, for example, to place ice or an ice cube in the liquid 102.

The generation of a cold droplet mist 100 is also possible in this way. On the other hand, the evaporative cold of the droplets, generated by the atomization, may in some cases be sufficient to trigger a subjective sensation of coldness and to elicit a cough in the test person.

For the purposes of the invention, it is possible to use a customary, commercially available appliance with ultrasound function and, optionally, a cooling function, into which appliance the storage vessel 103, with the liquid 102 that is to be atomized, is inserted for example. The storage vessel 103 can be coupled to the actual ultrasound transmitter/cooler via a further coupling medium, in particular a liquid. The storage vessel 103 can be designed as a disposable article, for example in a cost-effective manner from plastic, which article is formed accordingly. Thus, the storage vessel 103 can be disposed of after use. The ultrasound transmitter 101 and the optional Peltier cooling element 106 can be provided for mains operation or also for mobile battery operation.

The apparatus 10 further comprises a device 126 by which the droplet mist 100 can be aspirated by the test person and into which the test person exhales again or coughs, such that the device 126 is further provided for collecting the exhaled and/or coughed-up material. The device 126 is preferably designed as a disposable article, such that it can be disposed of after use in order to rule out the possibility of a further test person being infected by infectious material. The device 126 comprises a suction opening or a suction pipe 107 through which the droplet mist 100 is aspirated by the test person (arrow 115). The suction pipe 107 can be designed as a mouthpiece. In other designs, a mouthpiece can be plugged or screwed, for example, onto the suction pipe 107. The suction pipe 107 also serves to blow in the exhaled and/or coughed-up air flow after the droplet mist has been inhaled. To direct this air flow, here indicated by the arrow 116, a check valve 108 is provided between the suction pipe 107 and that end of the device 126 via which the generated droplet mist 100 flows into the device 126. With this check valve 108, the exhaled flow of air is diverted into a collecting vessel 109. This collecting vessel 109 can be in the form of, for example, a commercially available Eppendorf® filter vessel, which can be plugged onto a corresponding lateral opening or a lateral port 110 of the suction pipe 107. In this preferred embodiment, the collecting vessel 109 comprises a filter 111, in particular a filter frit, for example made of silicon oxide, on which the exhaled and/or coughed-up material is collected. So as not to generate any significant counterpressure during the blowing of the exhaled air into the device 126, the collecting vessel is provided with an outflow opening 112 through which the blown-in exhaled air can flow out in the arrow direction. After the test person has inhaled the droplet mist and the exhaled and/or coughed-up material has been blown back into the device 126, the collecting vessel 109 with the filter 111 contained therein can be removed in a simple manner and, in particular, the filter 111 can be removed for further analysis of the material and processed further.

The device 126 is connected to the device 114 (mist generator) in such a way that the droplet mist 100 generated can flow through the device 126. For this purpose, the device 126 can be plugged or screwed into the droplet mist outlet opening 105 via a connector piece 113 for example. For this purpose, a suitable seal or a thread can be provided in the region of the connection.

The connector piece 113 to the rear of the check valve 108 can, for example, be plugged directly into the droplet mist outlet opening 105, wherein further sealing means can be provided here to ensure a tight closure.

The check valve 108 is expediently realized as a simple mechanical check valve, such that the droplet mist 100 or the water droplet/air mixture can only be aspirated from the mist generator 114, which is formed in particular of the storage vessel 103, the ultrasound transmitter 101 and optionally the Peltier cooling element 106, and can no longer flow back. Such check valves 108 can be made of plastic, for example, and are available at low cost.

To prepare for taking a sample, the device 126 can first of all be connected to the device 114 or the storage vessel 103, before the coupling of the ultrasound and optionally the Peltier cooling means takes place, for example via an additional water reservoir as coupling medium. Commercially available atomizers can be used here. The process of inhaling the droplet mist 100 and blowing the exhaled flow of air into the device 126 can be repeated one or more times. Once the sampling is complete, the whole device 126 can be separated from the mist generator 114. The one or more collecting vessels 109 can, for example, be broken off or unscrewed, and the biological material contained therein, which may be bound to the filter 111, can undergo molecular biological analysis, for example with the aid of lab-on-a-chip analyses.

If the device 126 and optionally the storage vessel 103 are designed as disposable articles, then, after removal of the collecting vessels 109, the whole device 26 and optionally the storage vessel 103 can be discarded, since these parts may possibly be contaminated with pathogens.

These parts may also be stored on a temporary basis, for example with disinfectant solution, and later disposed of, for example by burning them.

If the storage vessel 103 is also designed as a disposable article, the check valve 108 does not have to be impermeable to microbes, since contamination by the disposable article is ruled out in any case. Therefore, the whole device 126 and also the storage vessel 103 can be manufactured as one or more inexpensive plastic parts that do not represent a significant cost factor. For example, the suction pipe 107, with the port 110 and the check valve 108 and also the connector part 113, can be manufactured as a one-piece article. It is also possible to combine these elements as a one-piece article with the storage vessel 103. This makes the use particularly easy since, in order to prepare to take a sample, all that needs to be done is to plug one or more collecting vessels 109 onto the one or more ports 110 and to insert the storage vessel 103 into the corresponding ultrasound/cooling appliance.

FIG. 2 shows a device 206 for the aspiration of the droplet mist and for collecting the exhaled/coughed-up material, the device 206 being provided for two collecting vessels 209. These collecting vessels 209, each with a filter 211, are plugged on via ports 210 located on opposite sides of the suction pipe 207. In other embodiments, a predetermined breaking point 210 can be provided instead of a port, such that the collecting vessels 209 can be broken off or twisted off after use, in order to be taken for further analysis. In other respects, the device 206 is comparable to the device 126 from FIG. 1, with a check valve 208 being provided in the interior of the device 206. With the region 213, the device 206 can be plugged or screwed into a droplet mist outlet opening of a storage vessel of a mist generator for the liquid that is to be atomized, wherein a corresponding thread can be provided.

The apparatuses described here serve to obtain a breath sample from a test person, wherein the droplet mist generated in the apparatuses, which preferably has a temperature below the ambient temperature, is inhaled and interacts with the surface of the test person's airways, wherein biological material located there is absorbed and at the same time a cough stimulus is generated, which has the effect that the water droplets charged with sample material are coughed up. By providing the substantially disposable apparatus with a check valve, the exhaled aerosol is forced through the preferably provided filters of the collecting vessels, such that the biological sample material is held back on the filters. The collecting vessels, with the filters thus charged, can be separated and, for example, taken for a molecular biological analysis and diagnosis process, preferably in lab-on-a-chip systems. These apparatuses and methods are particularly suitable for screening bacterial infections of the lungs, for example for tuberculosis screening. However, other diseases of the lungs, for example systemic diseases caused by bacteria, fungi or viruses or tumor diseases, can also be diagnosed and/or monitored using this method and the apparatuses described.

1. A method for obtaining a breath sample from a test person, comprising:
inhaling, by the test person, a droplet mist;
at least one of exhaling and coughing up material by the test person after inhaling the droplet mist; and
collecting the at least one of the exhaled and the coughed-up material as a breath sample.

2. The method as claimed in claim 1, wherein the droplet mist is colder than the ambient temperature.

3. The method as claimed in claim 1, further comprising:
generating the droplet mist with an ultrasonic atomizer.

4. The method as claimed in claim 1, further comprising: cooling the droplet mist using at least one of ice and at least one Peltier element.

5. The method as claimed in claim 1, further comprising:
conveying the at least one of the exhaled and the coughed-up material to at least one collecting vessel.

6. The method as claimed in claim 1, further comprising:
guiding the at least one of the exhaled and the coughed-up material through a filter; and
binding the at least one of the exhaled and the coughed-up material with the filter.

7. An apparatus for obtaining a breath sample from a test person, comprising:
a device configured to generate a droplet mist; and
a device configured to aspirate the droplet mist by the test person and to collect at least one of exhaled and coughed-up material from the test person.

8. The apparatus as claimed in claim 7, wherein the device configured to generate the droplet mist comprises:
a storage vessel configured to store a liquid that is to be atomized; and
an ultrasound transmitter.

9. The apparatus as claimed in claim 8, wherein the storage vessel is a disposable article.

10. The apparatus as claimed in claim 7, wherein the device configured to generate the droplet mist comprises:
a cooling device.

11. The apparatus as claimed in claim 7, wherein the device configured to aspirate comprises:
at least one check valve; and
at least one collecting vessel.

12. The apparatus as claimed in claim 11, wherein the at least one collecting vessel comprises;
at least one filter configured to bind the at least one of the exhaled and the coughed-up material.

13. The apparatus as claimed in claim 12, wherein the at least one comprises filter an SiO₂ frit.

14. The apparatus as claimed in claim 12, wherein the filter comprises a porous Teflon film.

15. The apparatus as claimed in claim 7, wherein the device configured to aspirate consists of one or more disposable articles.
